(19) United States
(12) Patent Application Publication
(10) Pub. No.: US 2022/0324808 A1
(43) Pub. Date: Oct. 13, 2022

(54) Co-crystal of Compound I Dihydrochloride and Preparation Method and Use Thereof

(71) Applicant: CRYSTAL PHARMACEUTICAL (SUZHOU) CO., LTD., Suzhou (CN)

(72) Inventors: Minhua CHEN, Suzhou (CN); Jing ZHANG, Suzhou (CN); Wenrui SHI, Suzhou (CN)

(73) Assignee: CRYSTAL PHARMACEUTICAL (SUZHOU) CO., LTD., Suzhou (CN)

(21) Appl. No.: 17/852,471

(22) Filed: Jun. 29, 2022

Related U.S. Application Data
(63) Continuation of application No. PCT/CN2020/141960, filed on Dec. 31, 2020.

(30) Foreign Application Priority Data
Jan. 3, 2020 (CN) 202010005187.X
Jan. 19, 2020 (CN) 202010062506.0

(51) Int. Cl. C07D 213/75

(52) U.S. Cl. CPC C07D 213/75; C07B 2200/13

(57) ABSTRACT

Provided are co-crystals of Compound I dihydrochloride and preparation methods thereof, pharmaceutical compositions containing the co-crystals, and uses of the co-crystals for preparing cardiac muscle myosin agonist drugs and drugs for treating heart failure. Compared with prior arts, the provided co-crystals of Compound I dihydrochloride have one or more improved properties, which solve the problems of prior arts and are of great value to the optimization and development of the drugs.

### CROSS-REFERENCE TO RELATED APPLICATIONS

This application is a continuation of International Application No. PCT/2020/141960, filed Dec. 31, 2020, which claims the benefit of priority to Chinese patent applications CN202010005187.X filed Jan. 3, 2020, and CN202010062506.0 filed Jan. 19, 2020. The contents of each Chinese patent application are incorporated herein by reference in their entireties.

### TECHNICAL FIELD

The present disclosure pertains to the field of chemical crystallography, particularly relates to co-crystals of Compound I dihydrochloride, preparation method and use thereof.

### BACKGROUND

Heart failure (HF) is a cardiac cycle disorder syndrome that caused by venous system blood sedimentation due to the dysfunction of systolic and/or diastolic function of the heart. Myocardial shrinkage reduction is the main sign of heart failure. The cardiac sarcomere is a highly ordered cytoskeletal structure composed of cardiac muscle myosin, actin and a set of regulatory proteins, with autorhythmicity, conductivity and contractility, and is the functional basis of systole and/or diastole. The cardiac muscle myosin, a molecular motor of the cytoskeleton, is a multifunctional protein that directly converts chemical energy into kinetic energy to provide power for systole.

Traditional drugs used to enhance myocardial contractility, such as β-adrenergic receptor agonists or angiotensin converting enzyme inhibitors, enhance myocardial contractility by increasing the concentration of Ca²⁺in cardiomyocytes. While these drugs can easily lead to life-threatening side effects of arrhythmia, tachycardia, increased myocardial oxygen consumption, etc. Cardiac muscle myosin agonists have enzymatic activity, which can improve the utilization of ATP, and directly regulate the activity of cardiac muscle myosin to improve the cardiac contractility and prolong cardiac contraction time.

Compound I (CK-1827452) is a cardiac muscle myosin agonist with the chemical name of methyl 4-[[2-fluoro-3-[N′-(6-methylpyridin-3-yl) ureido] phenyl]methyl] piperazine-1-carboxylate (Referred to as Compound I), and the structure is shown as the follows:

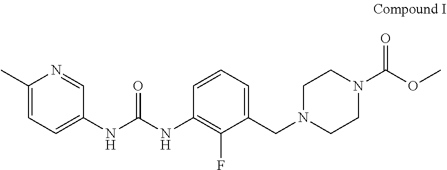

A crystalline form is a solid material whose constituents are arranged in a highly ordered microscopic structure, forming a crystal lattice that extends in all directions. Polymorphism refers to the phenomenon that a compound exists in more than one crystalline form. Different crystalline forms of drug substances have different physicochemical properties, which can affect drug's in vivo dissolution and absorption and will further affect drug's clinical efficacy and safety to some extent. In particular, for some poorly soluble solid dosage forms, crystalline forms can be crucial to the performance of drug product. Therefore, polymorphism is an important part of drug research and drug quality control.

According to FDA Regulatory Classification of Pharmaceutical Co-Crystals Guidance for Industry, pharmaceutical co-crystals are crystalline materials composed of two or more different molecules (one of which is the active pharmaceutical ingredient (API)) in a defined stoichiometric ratio within the same crystal lattice that are associated by nonionic and noncovalent bonds. Pharmaceutical co-crystals have provided opportunities for engineering solid-state forms beyond conventional solid-state forms of an API, such as salts and polymorphs. Pharmaceutical co-crystals can be tailored to enhance drug product bioavailability and stability and to enhance the processability of APIs during drug production process.

Compound I dihydrochloride crystalline Form A, Form B and Form C were disclosed in WO2014152270A1, and the specification also disclosed the following: when Form A hydrate was heated to above about 75° C., the material converted to Form B. When the material was cooled down to ambient conditions, Form B absorbed water from the atmosphere and converted back to the hydrate Form A. When Form A was exposed to 5% relative humidity (RH), the material converted to Form C. When the material was exposed to 15% RH and higher, Form C resorbed water from the environment and converted to the hydrate Form A. Although Form A shows certain advantages as Form B and Form C convert easily to Form A, the dynamic vapor sorption result shows that Form A has a total weight gain of about 0.55 wt % between about 40% RH and about 95% RH and a weight loss of about 2.7 wt % between about 30% RH and about 5% RH along with crystalline form conversion. The poor humidity stability of Form A is an inevitable risk in its industrial production.

WO2020014406A1 disclosed several crystalline forms, which are Compound I dihydrochloride crystalline forms O-S1, O-S2, O-S3, O-S4, O-S5 and amorphous. Crystalline forms O-S1, O-S2, O-S3, O-S4 and O-S5 are all solvates prepared in acid solvents.

Amorphous is thermodynamically unstable because of the disordered arrangement of molecules. Amorphous solids are in a high-energy state and usually have poor stability. Amorphous drug substance is prone to crystal transformation during the manufacturing process and storage which will lead to an inconsistency of drug bioavailability, dissolution rate, etc., resulting in changes in the drug's clinical efficacy. In addition, the preparation of amorphous is usually a rapid kinetic solid precipitation process, which easily leads to excessive residual solvents, and its particle properties are difficult to be controlled by the process, resulting in great challenges in the practical application.

In order to overcome the disadvantages of prior arts, the inventors of the present disclosure surprisingly discovered the co-crystals of Compound I dihydrochloride with fumaric acid and Compound I dihydrochloride with tartaric acid, which have advantages in physiochemical properties, formulation processability, bioavailability, etc., for example, the co-crystals have advantages in at least one aspect of melting point, solubility, hygroscopicity, purification ability, stability, adhesiveness, compressibility, flowability, in vitro and in vivo dissolution, bioavailability, etc. In particular, the co-crystals have good stability, low hygroscopicity, good compressibility, low adhesiveness, and good formulation dissolution, which could solve the problems existing in prior arts and are of great significance for the development of drugs containing Compound I.

### SUMMARY

The present disclosure is to provide co-crystals of Compound I dihydrochloride, preparation method and use thereof.

According to the objective of the present disclosure, a co-crystal Form CSI of Compound I dihydrochloride with fumaric acid is provided (hereinafter referred to as Form CSI).

In one aspect provided herein, the molar ratio of Compound I dihydrochloride and fumaric acid in Form CSI is 2:1.

Furthermore, the X-ray powder diffraction pattern of Form CSI comprises characteristic peaks at 2theta values of 6.2°±0.2°, 17.4°±0.2° and 25.8°±0.2° using CuKα radiation.

Furthermore, the X-ray powder diffraction pattern of Form CSI comprises one or two or three characteristic peaks at 2theta values of 12.6°±0.2°, 19.6°±0.2° and 23.5°±0.2°. Preferably, the X-ray powder diffraction pattern of Form CSI comprises three characteristic peaks at 2theta values of 12.6°±0.2°, 19.6°±0.2° and 23.5°±0.2° using CuKα radiation.

Furthermore, the X-ray powder diffraction pattern of Form CSI comprises one or two or three characteristic peaks at 2theta values of 15.4°±0.2°, 21.1°±0.2° and 26.3°±0.2°. Preferably, the X-ray powder diffraction pattern of Form CSI comprises three characteristic peaks at 2theta values of 15.4°±0.2°, 21.1°±0.2° and 26.3°±0.2° using CuKα radiation.

In another aspect provided herein, the X-ray powder diffraction pattern of Form CSI comprises three or four or five or six or seven or eight or nine or ten or eleven characteristic peaks at 2theta values at 6.2°±0.2°, 17.4°±0.2°, 25.8°±0.2°, 12.6°±0.2°, 19.6°±0.2°, 23.5°±0.2°, 16.7±0.2°, 24.8±0.2°, 15.4°±0.2°, 21.1°±0.2° and 26.3°±0.2° using CuKα radiation.

Without any limitation being implied, the X-ray powder diffraction pattern of Form CSI is substantially as depicted in FIG. 1.

Without any limitation being implied, the Thermo Gravimetric Analysis (TGA) curve of Form CSI is substantially as depicted in FIG. 2, which shows about 2.9% weight loss when heated to 130° C.

Without any limitation being implied, Form CSI is a co-crystal hydrate.

According to the objective of the present disclosure, a process for preparing Form CSI is also provided. The process comprises: adding Compound I dihydrochloride solid and fumaric acid solid in a solvent mixture of a nitrile and water, stirring to obtain Form CSI.

Furthermore, molar ratio of said Compound I dihydrochloride solid and fumaric acid solid is 1:3-2:1, said nitrile is acetonitrile, volume ratio of acetonitrile and water in said solvent mixture is 9:1.

Form CSI of the present disclosure has the following advantages:

(1) Compared with prior arts, Form CSI of the present disclosure has better in vitro dissolution. In pH6.8 phosphate buffered saline (PBS), the dissolution of Form CSI drug product is higher than that of Form A in WO2014152270A1.

Drugs with different crystalline forms may lead to different in vivo dissolution, which directly affects the in vivo absorption, distribution, metabolism and excretion of the drug, and ultimately leads to different clinical efficacy due to their different bioavailability. Drug dissolution and dissolution rate are prerequisites for drug absorption. Good in vitro dissolution may lead to higher in vivo absorption, and better in vivo exposure, thereby improving drug's bioavailability and efficacy.

(2) Form CSI drug substance of the present disclosure has good stability itself and in drug product. Crystalline state of Form CSI drug substance doesn't change for at least six months when stored under the condition of 25° C./60% RH. The chemical purity is above 99.6% and remains substantially unchanged during storage. These results show that Form CSI drug substance has good stability under long term condition, which is beneficial to drug storage.

Meanwhile, crystalline state of Form CSI drug substance doesn't change for at least six months when stored under the condition of 40° C./75% RH. The crystalline state of Form CSI drug substance doesn't change for at least one month when stored under the condition of 60° C./75% RH. The chemical purity is above 99.6% and remains substantially unchanged during storage. After Form CSI is mixed with the excipients to form a drug product and stored under the condition of 40° C./75% RH, the crystalline state of Form CSI drug product doesn't change for at least three months and the chemical purity remains substantially unchanged. These results show that Form CSI drug substance has good stability under accelerated and stress conditions both itself and in drug product. Drug substance will go through high temperature and high humidity conditions caused by different seasons, regional climate and weather during storage, transportation and manufacturing processes. Therefore, good stability under accelerated and stress conditions is of great importance to the drug development. Form CSI drug substance and product have good stability under these stress conditions, which is beneficial to avoid the influence on drug quality when isn't stored in the conditions recommended in the label.

Meanwhile, Form CSI has good mechanical stability. Form CSI has good physical stability after grinding. Grinding and pulverization are often required in the drug manufacturing process. Good physical stability of the drug substance can reduce the risk of crystallinity decrease and crystal transformation during the drug manufacturing process. Form CSI has good physical stability under different pressures, which is beneficial to keep crystalline form unchanged during tableting process.

Form CSI has good stability under different humidity conditions, the crystalline state does not change after DVS test with a humidity range of 0-95% RH. In particular, the crystalline state of Form CSI remains unchanged under low humidity conditions. The crystalline state of prior arts changed under low humidity conditions.

Crystalline form transformation can lead to changes in the absorption of the drug, affect bioavailability, and even cause toxicity and side effects. Good chemical stability ensures that no impurity would be generated during storage. Form CSI has good physical and chemical stability, ensuring consistent and controllable quality of the drug substance and drug product, and minimizing quality changes, bioavailability changes, toxicity and side effects caused by crystal transformation or impurity generation.

(3) Compared with prior arts, Form CSI of the present disclosure shows lower adhesiveness. Adhesiveness evaluation results indicate that adhesion quantity of Form CSI is remarkably lower than that of prior art forms. Superior adhesiveness of Form CSI can effectively improve the adhesion to roller and tooling during dry-granulation and compression process, which is also beneficial to improve product appearance and weight variation. In addition, superior adhesiveness of Form CSI can reduce the agglomeration of drug substance, which is beneficial to the dispersion of drug substance with excipients, improve the blend uniformity of the mixing of materials, and ultimately improves the quality uniformity of the product.

According to the objective of the present disclosure, a co-crystal Form CSIII of Compound I dihydrochloride with tartaric acid is provided (hereinafter referred to as Form CSIII).

In one aspect provided herein, the molar ratio of Compound I dihydrochloride and tartaric acid in Form CSIII is 1:1.

Furthermore, the X-ray powder diffraction pattern of Form CSIII comprises characteristic peaks at 2theta values of 17.2°±0.2°, 20.2°±0.2° and 25.7°±0.2° using CuKα radiation.

Furthermore, the X-ray powder diffraction pattern of Form CSIII comprises one or two or three characteristic peaks at 2theta values of 19.4°±0.2°, 24.4°±0.2° and 30.6°±0.2°. Preferably, the X-ray powder diffraction pattern of Form CSI comprises three characteristic peaks at 2theta values of 19.4°±0.2°, 24.4°±0.2° and 30.6°±0.2° using CuKα radiation.

Furthermore, the X-ray powder diffraction pattern of Form CSIII comprises one or two or three characteristic peaks at 2theta values of 18.0°±0.2°, 14.7°±0.2° and 21.3°±0.2°. Preferably, the X-ray powder diffraction pattern of Form CSI comprises three characteristic peaks at 2theta values of 18.0°±0.2°, 14.7°±0.2° and 21.3°±0.2° using CuKα radiation.

In another aspect provided herein, the X-ray powder diffraction pattern of Form CSIII comprises three or four or five or six or seven or eight or nine or ten or eleven characteristic peaks at 2theta values at 17.2°±0.2°, 20.2°±0.2°, 25.7°±0.2°, 19.4°±0.2°, 24.4°±0.2°, 30.6°±0.2°, 18.0°±0.2°, 14.7°±0.2°, 21.3°±0.2°, 16.4°±0.2° and 23.3°±0.2° using CuKα radiation.

Without any limitation being implied, the X-ray powder diffraction pattern of Form CSIII is substantially as depicted in FIG. 8.

Without any limitation being implied, the TGA curve of Form CSIII is substantially as depicted in FIG. 9, which shows 0.3% weight loss when heated to 100° C.

Without any limitation being implied, Form CSIII is an anhydrate.

According to the objective of the present disclosure, a process for preparing Form CSIII is also provided. The process comprises: adding Compound I dihydrochloride solid and tartaric acid solid in an ester, slurring and separating to obtain co-crystal of Compound I dihydrochloride with tartaric acid.

Furthermore, molar ratio of said Compound I dihydrochloride solid and tartaric acid solid is 1:3-1:1, said ester is ethyl acetate, said slurring temperature is preferably room temperature.

Furthermore, said tartaric acid is L- tartaric acid, D-tartaric acid, or DL- tartaric acid, preferably L- tartaric acid.

Form CSIII of the present disclosure has the following advantages:

(1) Form CSIII drug substance of the present disclosure has good stability itself and in drug product. Crystalline state of Form CSIII drug substance doesn't change for at least three months when stored under the condition of 25° C./60% RH. The chemical purity is above 99.3% and remains substantially unchanged during storage. These results show that Form CSIII drug substance has good stability under long term condition, which is beneficial to drug storage.

Meanwhile, crystalline state of Form CSIII drug substance doesn't change for at least three months when stored under the condition of 40° C./75% RH with sealed condition. The crystalline state of Form CSIII drug substance doesn't change for at least three months when stored under the condition of 60° C./75% RH with sealed condition. The chemical purity is above 99.3% and remains substantially unchanged during storage. After Form CSIII is mixed with the excipients to form a drug product and stored under the condition of 40° C.±2° C./75%±5% RH, the crystalline state of Form CSIII drug product doesn't change for at least three months and the chemical purity remains substantially unchanged. These results show that Form CSIII drug substance has good stability under accelerated and stress conditions both itself and in drug product. Drug substance will go through high temperature and high humidity conditions caused by different seasons, regional climate and weather during storage, transportation and manufacturing processes. Therefore, good stability under accelerated and stress conditions is of great importance to the drug development. Form CSIII drug substance and product have good stability under these stress conditions, which is beneficial to avoid the influence on drug quality when isn't stored in the conditions recommended in the label.

Meanwhile, the crystalline form CSIII has good high-temperature stability, and has about 0.3% weight loss when heated to 100° C.

Meanwhile, Form CSIII has good mechanical stability. Form CSIII drug substance has good physical stability after grinding. Grinding and pulverization are often required in the drug manufacturing process. Good physical stability of the drug substance can reduce the risk of crystallinity decrease and crystal transformation during the drug manufacturing process. Form CSIII has good physical stability under different pressures, which is beneficial to keep crystalline form unchanged during tableting process.

Crystalline form transformation can lead to changes in the absorption of the drug, affect bioavailability, and even cause toxicity and side effects. Good chemical stability ensures that no impurity would be generated during storage. Form CSIII has good physical and chemical stability, ensuring consistent and controllable quality of the drug substance and drug product, and minimizing quality changes, bioavailability changes, toxicity and side effects caused by crystal transformation or impurity generation.

Further, Form CSIII provided by the present disclosure also has the following advantages:

(1) Compared with prior arts, Form CSIII of the present disclosure shows lower adhesiveness. Adhesiveness evaluation results indicate that adhesion quantity of Form CSIII is remarkably lower than that of prior art forms. Superior adhesiveness of Form CSIII can effectively improve the adhesion to roller and tooling during dry-granulation and compression process, which is also beneficial to improve product appearance and weight variation. In addition, superior adhesiveness of Form CSIII can reduce the agglomeration of drug substance, which is beneficial to the dispersion of drug substance with excipients, improve the blend uniformity of the mixing of materials, and ultimately improves the quality uniformity of the product.

(2) Compared with prior arts, Form CSIII of the present disclosure has better compressibility. Failure in hardness/friability test and tablet crack issue can be avoided due to better compressibility of Form CSIII, making the preparation process more reliable, improving product appearance, and promoting product quality. Better compressibility can increase the compression rate, thus further increases the efficiency of process and reduces the cost of compressibility improving excipients.

According to the objective of the present disclosure, a pharmaceutical composition is provided. Said pharmaceutical composition comprises a therapeutically effective amount of Form CSI or Form CSIII, and pharmaceutically acceptable excipients.

Furthermore, a method of agonizing cardiac muscle myosin is provided. Said method comprises administering to a subject in need thereof a therapeutically effective amount of Form CSI, Form CSIII, or the combination thereof.

Furthermore, a method for treating heart failure is provided. Said method comprises administering to a subject in need thereof a therapeutically effective amount of Form CSI, Form CSIII, or the combination thereof.

In the present disclosure, said “stirring” is accomplished by using a conventional method in the field such as magnetic stirring or mechanical stirring and the stirring speed is 50 to 1800 r/min, preferably the magnetic stirring speed is 300 to 900 r/min and mechanical stirring speed is 100 to 300 r/min.

Said “separation” is accomplished by using a conventional method in the field such as centrifugation or filtration. The operation of “centrifugation” is as follows: the sample to be separated is placed into the centrifuge tube, and then centrifuged at a rate of 10000 r/min until the solid all sink to the bottom of the tube.

Said “characteristic peak” refers to a representative diffraction peak used to distinguish crystals, which usually can have a deviation of ±0.2° using CuKα radiation.

In the present disclosure, “crystal” or “crystalline form” refers to the crystal or the crystalline form being identified by the X-ray diffraction pattern shown herein. Those skilled in the art are able to understand that the experimental errors depend on the instrument conditions, the sample preparation and the purity of samples. The relative intensity of the diffraction peaks in the X-ray diffraction pattern may also vary with the experimental conditions. Therefore, the order of the diffraction peak intensities cannot be regarded as the sole or decisive factor. In fact, the relative intensity of the diffraction peaks in the X-ray powder diffraction pattern is related to the preferred orientation of the crystals, and the diffraction peak intensities shown herein are illustrative and not for absolute comparison. Thus, it will be understood by those skilled in the art that a crystalline form of the present disclosure is not necessarily to have exactly the same X-ray diffraction pattern of the example shown herein. Any crystalline forms whose X-ray diffraction patterns have the same or similar characteristic peaks should be within the scope of the present disclosure. Those skilled in the art can compare the patterns shown in the present disclosure with that of an unknown crystalline form in order to identify whether these two groups of patterns reflect the same or different crystalline forms.

In some embodiments, Form CSI and Form CSIII of the present disclosure are pure and substantially free of any other crystalline forms. In the present disclosure, the term “substantially free” when used to describe a novel crystalline form, it means that the content of other crystalline forms in the novel crystalline form is less than 20% (w/w), specifically less than 10% (w/w), more specifically less than 5% (w/w) and furthermore specifically less than 1% (w/w).

In the present disclosure, the term “about” when referring to a measurable value such as weight, time, temperature, and the like, is meant to encompass variations of ±10%, ±5%, ±1%, ±0.5%, or even ±0.1% of the specified amount.

### BRIEF DESCRIPTION OF THE DRAWINGS

FIG. 1 shows an XRPD pattern of Form CSI in Example 1

FIG. 2 shows a TGA curve of Form CSI in Example 1

FIG. 3 shows an XRPD pattern of Form CSI in Example 2

FIG. 4 shows a TGA curve of Form CSI in Example 2

FIG. 5 shows an XRPD pattern overlay of Form CSI before and after storage (from top to bottom: initial, stored at 25° C./60% RH (sealed) for six months, stored at 25° C./60% RH (open) for six months, stored at 40° C./75% RH (sealed) for six months, stored at 40° C./75% RH (open) for six months, stored at 60° C./75% RH (sealed) for one month).

FIG. 6 shows a DVS plot of Form CSI

FIG. 7 shows an XRPD pattern overlay of Form CSI before and after DVS test (top: before DVS, bottom: after DVS)

FIG. 8 shows an XRPD pattern of Form CSIII in Example 6

FIG. 9 shows a TGA curve of Form CSIII in Example 6

FIG. 10 shows an XRPD pattern of Form CSIII in Example 7

FIG. 11 shows a TGA curve of Form CSIII in Example 7

FIG. 12 shows a DSC curve of Form CSIII in Example 7

FIG. 13 shows an XRPD pattern overlay of Form CSIII before and after storage (from top to bottom: initial, stored at 25° C./60% RH (sealed with desiccant) for three months, stored at 25° C./60% RH (open) for three months, stored at 40° C./75% RH (sealed with desiccant) for three months, stored at 60° C./75% RH (sealed with desiccant) for three month).

FIG. 14 shows an XRPD pattern overlay of Form A before and after grinding (top:

before grinding, bottom: after grinding).

FIG. 15 shows an XRPD pattern overlay of Form CSI before and after grinding (top: before grinding, bottom: after grinding).

FIG. 16 shows an XRPD pattern overlay of Form CSIII before and after grinding (top: before grinding, bottom: after grinding).

FIG. 17 shows an XRPD pattern overlay of Form CSI tableting under different pressure (from top to bottom: 20 kN, 10 kN, 5 kN, 0 kN).

FIG. 18 shows an XRPD pattern overlay of Form CSIII tableting under different pressure (from top to bottom: 20 kN, 10 kN, 5 kN, 0 kN).

FIG. 19 shows an XRPD pattern overlay of Form CSI and Form CSI drug product (from top to bottom: excipients, Form CSI drug product, and Form CSI).

FIG. 20 shows an XRPD pattern overlay of Form CSIII and Form CSIII drug product (from top to bottom: excipients, Form CSIII drug product, and Form CSIII).

FIG. 21 shows an XRPD pattern overlay of Form CSI drug product stored under different conditions (from top to bottom: initial, stored under 40° C.±2° C./75%±5% RH (sealed with 1 g desiccant) for 3 months)

FIG. 22 shows an XRPD pattern overlay of Form CSIII drug product stored under different conditions (from top to bottom: initial, stored under 40° C.±2° C./75%±5% RH (sealed with 1 g desiccant) for 3 months)

FIG. 23 shows a dissolution curve of Form CSI drug product and Form A drug product in pH6.8 PBS.

### DETAILED DESCRIPTION

The present disclosure is further illustrated by the following examples which describe the preparation and use of the crystalline forms of the present disclosure in detail. It is obvious to those skilled in the art that changes in the materials and methods can be accomplished without departing from the scope of the present disclosure.

The abbreviations used in the present disclosure are explained as follows

XRPD: X-ray Powder Diffraction

TGA: Thermo Gravimetric Analysis

DSC: Differential Scanning calorimetry

HPLC: High Performance Liquid Chromatography

IC: Ion Chromatography

¹H NMR: Proton Nuclear Magnetic Resonance

DVS: Dynamic Vapor Sorption

Instruments and methods used for data collection

The X-ray powder diffraction patterns for the use of stability characterization of

Form CSI were acquired by a Bruker D8 DISCOVER X-ray powder diffractometer. The parameters of the X-ray powder diffraction method of the present disclosure are as follows:

X-Ray source: Cu, Kα

Kα1 (Å): 1.54060; Kα2 (Å): 1.54439

Kα2/Kα1 intensity ratio: 0.50

Voltage: 40 (kV)

Current: 40 (mA)

Scan range (2θ): from 4.0 degree to 40.0 degree

Except the samples tested by the Bruker D8 DISCOVER X-ray powder diffractometer, the other X-ray powder diffraction patterns were acquired by a Bruker D2 PHASER X-ray powder diffractometer. The parameters of the X-ray powder diffraction method of the present disclosure are as follows:

X-Ray source: Cu, Kα

Kα1 (Å): 1.5406; Kα2 (Å): 1.54439

Kα2/Kα1 intensity ratio: 0.50

Voltage: 30 (kV)

Current: 10 (mA)

Scan range (2θ): from 3.0 degree to 40.0 degree

Thermo gravimetric analysis (TGA) data in the present disclosure were acquired by a TA Q500. The parameters of the TGA method of the present disclosure are as follows:

Heating rate: 10° C./min

Purge gas: nitrogen

Differential scanning calorimetry (DSC) data in the present disclosure were acquired by a TA Q2000. The parameters of the DSC method of the present disclosure are as follows:

Heating rate: 10° C./min

Purge gas: nitrogen

Dynamic Vapor Sorption (DVS) was measured via an SMS (Surface Measurement Systems Ltd.) intrinsic DVS instrument. Typical Parameters for DVS test are as follows:

Temperature: 25° C.

Gas and flow rate: nitrogen, 200 mL/min

Rate of mass change: 0.002%/min

RH range: 0% RH to 95% RH

Proton nuclear magnetic resonance spectrum data (¹H NMR) were collected from a Bruker Avance II DMX 400M HZ NMR spectrometer. 1-5 mg of sample was weighed and dissolved in 0.5 mL of deuterated dimethyl sulfoxide to obtain a solution with a concentration of 2-10 mg/mL.

The assay of Compound I in Form CSIII of the present disclosure is detected by

HPLC and the parameters are shown in Table 1.

TABLE 1

| HPLC | Agilent 1290 with DAD detector | |
|---|---|---|
| Column | Agilent ZORBAX Eclipse Plus C18 Rapid | |
| | Resolution HD, 2.1*50 mm, 1.8 μm | |
| Mobile Phase | A: 0.05% H₃PO₄ aqueous solution | |
| | (pH 6.8, triethylamine) | |
| | B: Acetonitrile | |
| | Time (min) | % B |
| Gradient | 0.0 | 30.0 |
| | 1.0 | 30.0 |
| | 3.0 | 40.0 |
| | 6.0 | 70.0 |
| | 6.1 | 30.0 |
| | 8.0 | 30.0 |

| | |
|---|---|
| Run Time | 8.0 min |
| Post Time | 0.0 min |
| Speed | 0.5 mL/min |
| Injection Volume | 1 μL |
| Detection | UV at 254 nm |
| Wavelength | |
| Column Temperature | 40° C. |
| Sample Temperature | Room temperature |
| Diluent | H₂O |

The assay of tartaric acid in Form CSIII of the present disclosure is detected by HPLC and the parameters are shown in Table 2.

TABLE 2

| HPLC | Agilent 1260 with VWD detector | |
|---|---|---|
| Column | Ultimate LP-C18, 250*4.6 mm, 5 μm | |
| Mobile Phase | A: ACN: H₂O (pH = 3.0, H₃PO₄) = 5:95 | |
| | B: ACN | |
| | Time(min) | % B |
| Gradient | 0.0 | 0.0 |
| | 10.0 | 0.0 |
| | 15.0 | 70.0 |
| | 25.0 | 70.0 |
| | 26.0 | 0.0 |
| | 40.0 | 0.0 |

| | |
|---|---|
| Run Time | 40.0 min |
| Post Time | 0.0 min |
| Speed | 0.8 mL/min |
| Injection Volume | 2 μL |
| Detection Wavelength | UV at 210 nm |
| Column Temperature | 30° C. |
| Sample Temperature | Room temperature |
| Diluent | H₂O |

The assay of chloride ion in Form CSIII of the present disclosure is detected by IC, and the parameters are shown in Table 3.

TABLE 3

| | |
|---|---|
| IC | Thermo Fisher Dionex Aquion |
| Column | Thermo Dionex IonPac AS22, 4 × 250 mm, |
| | 6.0 μm |
| Mobile Phase | 4.5 mM Na₂CO₃/1.4 mM NaHCO₃ |
| Injection Volume | 25 μL |
| Flow rate | 1.0 mL/min |
| Conductivity Cell | 35° C. |
| Temperature | |
| Column Temperature: | 30° C. |
| Suppressor Current | 31 mA |
| Running Time | 8 min |

The parameters for related substance detection in the present disclosure are shown in Table 4.

TABLE 4

| HPLC | Agilent 1290 with DAD detector | |
|---|---|---|
| Column | Agilent ZORBAX Eclipse Plus C18 Rapid | |
| | Resolution HD, 2.1*50 mm, 1.8 μm | |
| Mobile Phase | A: 0.05% H₃PO₄ aqueous solution | |
| | (pH 6.8, triethylamine) | |
| | B: Acetonitrile | |
| | Time (min) | % B |
| Gradient | 0.0 | 30.0 |
| | 1.0 | 30.0 |
| | 3.0 | 40.0 |
| | 6.0 | 70.0 |
| | 6.1 | 30.0 |
| | 8.0 | 30.0 |

| | |
|---|---|
| Run Time | 8 min |
| Post Time | 0 min |
| Speed | 0.5 mL/min |
| Injection Volume | 2 μL |
| Detection Wavelength | UV, 254 nm |
| Column Temperature | 40° C. |
| Sample Temperature | Room temperature |
| Diluent | 80% acetonitrile aqueous solution |

The parameters for formulation dissolution detection in the present disclosure are shown in Table 5.

TABLE 5

| HPLC | Agilent 1290 with DAD detector | |
|---|---|---|
| Column | Agilent ZORBAX Eclipse Plus C18 Rapid | |
| | Resolution HD, 2.1*50 mm, 1.8 μm | |
| Mobile Phase | A: 0.05% H₃PO₄ aqueous solution (pH 6.8, | |
| | triethylamine) | |
| | B: Acetonitrile | |
| | Time (min) | % B |
| Gradient | 0.0 | 30.0 |
| | 1.0 | 30.0 |
| | 3.0 | 40.0 |
| | 6.0 | 70.0 |
| | 6.1 | 30.0 |
| | 8.0 | 30.0 |

| | |
|---|---|
| Run Time | 8 min |
| Post Time | 0 min |
| Speed | 0.5 mL/min |
| Injection Volume | 2 μL |
| Detection Wavelength | UV, 254 nm |
| Column Temperature | 40° C. |
| Sample Temperature | Room temperature |
| Diluent | 80% acetonitrile aqueous solution |

Unless otherwise specified, the following examples were conducted at room temperature. Said “room temperature” is not a specific temperature, but a temperature range of 10-30° C.

According to the present disclosure, Compound I dihydrochloride as a raw material is solid (crystalline and amorphous), semisolid, wax, oil, liquid form or solution. Preferably, Compound I dihydrochloride as a raw material is a solid.

Raw materials of Compound I dihydrochloride used in the following examples were prepared by known methods in prior arts, for example, the method disclosed in WO2014152270A1.

### EXAMPLES

### Example 1: Preparation of Form CSI

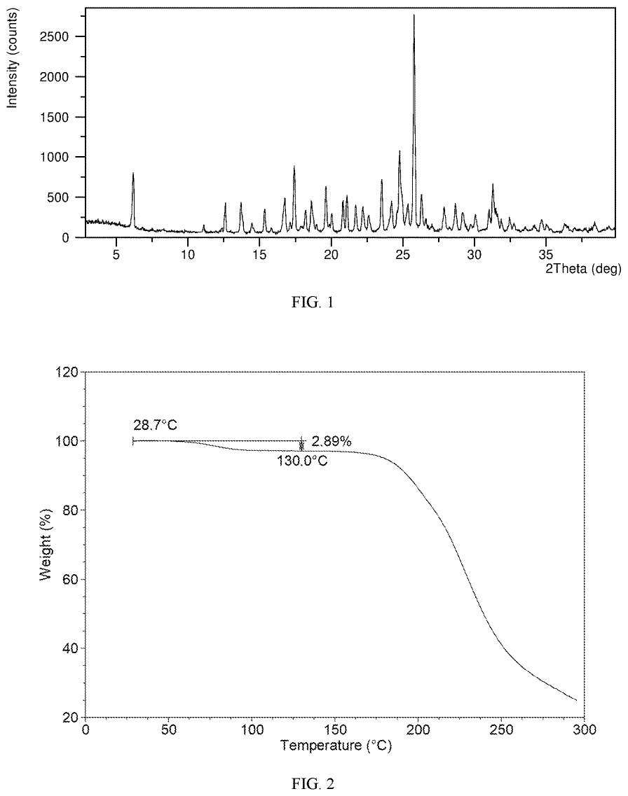

98.7 mg of Compound I dihydrochloride, 44.5 mg of fumaric acid, and 5.0 mL of acetonitrile/water (9:1, v/v) were mixed and the obtained suspension was stirred at room temperature for 13 days. The solid was separated and dried under vacuum at 25° C. for 40 minutes. The obtained solid is Form CSI and the XRPD pattern of Form CSI is substantially as depicted in FIG. 1, and the XRPD data are listed in Table 6.

The TGA curve of Form CSI shows about 2.9% weight loss when heated to 130° C., which is substantially as depicted in FIG. 2.

The ¹H NMR results show that the molar ratio of Compound I dihydrochloride and fumaric acid in Form CSI is 2:1 and the specific data are: ¹H NMR (400 MHz, DMSO-d6) δ 11.13 (s, 1H), 10.94 (s, 1H), 9.35 (d, J=2.3 Hz, 1H), 8.92 (d, J=2.5 Hz, 1H), 8.19 (ddd, J=16.2, 8.4, 2.1 Hz, 2H), 7.78 (d, J=8.8 Hz, 1H), 7.48-7.34 (m, 1H), 7.26 (t, J=8.0 Hz, 1H), 6.63 (s, 1H), 4.39 (s, 2H), 2.65 (s, 3H).

TABLE 6

| 2θ (°) | d spacing (Å) | Relative Intensity % |
|---|---|---|
| 6.18 | 14.30 | 25.47 |
| 11.10 | 7.97 | 2.97 |
| 12.60 | 7.02 | 13.10 |
| 13.70 | 6.46 | 13.53 |
| 14.47 | 6.12 | 3.44 |
| 15.35 | 5.77 | 10.35 |
| 15.82 | 5.60 | 1.97 |
| 16.76 | 5.29 | 15.75 |
| 17.42 | 5.09 | 29.76 |
| 18.21 | 4.87 | 10.04 |
| 18.62 | 4.76 | 13.88 |
| 19.63 | 4.52 | 20.90 |
| 20.03 | 4.43 | 8.54 |
| 20.82 | 4.27 | 14.01 |
| 21.10 | 4.21 | 16.57 |
| 21.71 | 4.09 | 12.25 |
| 22.21 | 4.00 | 11.74 |
| 22.62 | 3.93 | 7.60 |
| 23.52 | 3.78 | 24.00 |
| 24.20 | 3.68 | 13.54 |
| 24.77 | 3.59 | 37.26 |
| 25.35 | 3.51 | 12.74 |
| 25.78 | 3.46 | 100.00 |
| 26.31 | 3.39 | 17.25 |
| 27.88 | 3.20 | 11.11 |
| 28.67 | 3.11 | 13.16 |
| 29.18 | 3.06 | 8.59 |
| 30.06 | 2.97 | 8.21 |
| 31.01 | 2.88 | 10.27 |
| 31.28 | 2.86 | 22.34 |
| 32.46 | 2.76 | 6.26 |
| 33.53 | 2.67 | 1.85 |
| 34.17 | 2.62 | 2.53 |
| 34.68 | 2.59 | 5.54 |
| 36.32 | 2.47 | 3.18 |
| 38.39 | 2.34 | 3.20 |

### Example 2: Preparation of Form CSI

9.9 mg of Compound I dihydrochloride, 5.1 mg of fumaric acid, and 0.5 mL of acetonitrile/water (9:1, v/v) were mixed and the obtained suspension was stirred at room temperature for 21 days. The solid was separated to obtain a crystalline solid.

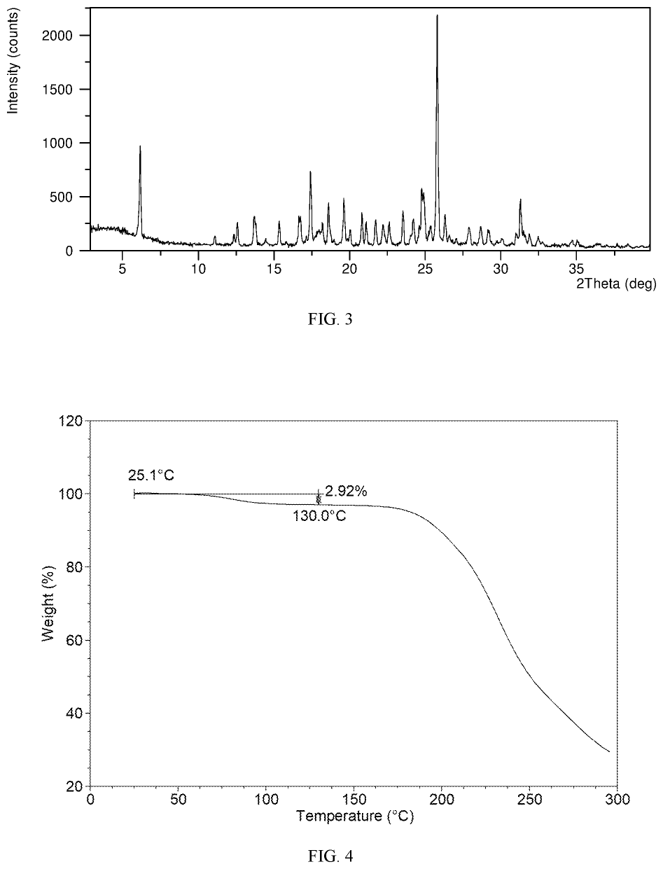

The crystalline solid was confirmed to be Form CSI, and the XRPD pattern of Form CSI is substantially as depicted in FIG. 3, and the XRPD data are listed in Table 7.

The TGA curve of Form CSI shows about 2.9% weight loss when heated to 130° C., which is substantially as depicted in FIG. 4.

TABLE 7

| 2θ (°) | d spacing (Å) | Relative Intensity % |
|---|---|---|
| 6.17 | 14.33 | 38.36 |
| 11.10 | 7.97 | 3.74 |
| 12.59 | 7.03 | 9.48 |
| 13.70 | 6.46 | 12.22 |
| 15.35 | 5.77 | 10.25 |
| 16.66 | 5.32 | 12.47 |
| 17.42 | 5.09 | 31.38 |
| 18.21 | 4.87 | 8.55 |
| 18.61 | 4.77 | 17.93 |
| 19.63 | 4.52 | 20.48 |
| 20.04 | 4.43 | 6.73 |
| 20.82 | 4.27 | 14.23 |
| 21.10 | 4.21 | 9.88 |
| 21.72 | 4.09 | 10.56 |
| 22.21 | 4.00 | 8.93 |
| 22.61 | 3.93 | 10.11 |
| 23.53 | 3.78 | 15.05 |
| 24.21 | 3.68 | 11.52 |
| 24.77 | 3.59 | 24.47 |
| 24.89 | 3.58 | 22.76 |
| 25.35 | 3.51 | 8.57 |
| 25.78 | 3.46 | 100.00 |
| 26.31 | 3.39 | 13.45 |
| 27.05 | 3.30 | 3.16 |
| 27.89 | 3.20 | 8.02 |
| 28.66 | 3.11 | 8.31 |
| 29.18 | 3.06 | 6.53 |
| 30.08 | 2.97 | 3.32 |
| 31.03 | 2.88 | 5.13 |
| 31.28 | 2.86 | 19.75 |
| 31.89 | 2.81 | 5.40 |
| 32.46 | 2.76 | 3.99 |
| 34.69 | 2.59 | 2.36 |
| 35.06 | 2.56 | 2.29 |
| 36.40 | 2.47 | 1.22 |
| 38.38 | 2.35 | 1.19 |

### Example 3: Preparation of Form CSI

423.9 mg of Compound I dihydrochloride solid, 238.6 mg of fumaric acid and 10 mL of acetonitrile/water (9:1, v:v) were mixed, and stirred at room temperature for 1 day. Then 5 mL of acetonitrile/water (9:1, v:v) was added into the system and the system was further stirred for 1 day. The solid was separated and dried under vacuum at 25° C. for 50 minutes to obtain Form CSI.

### Example 4: Stability of Form CSI

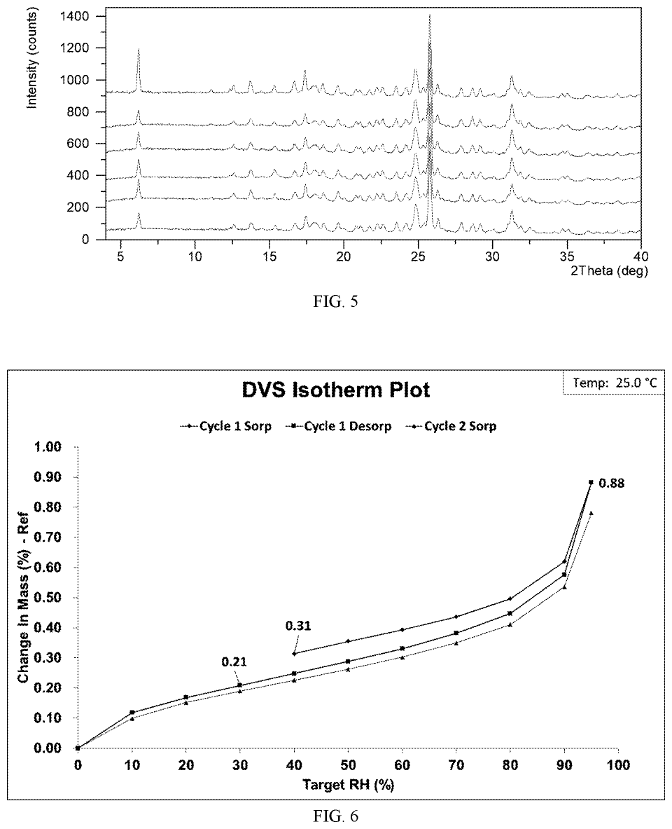

Approximately 5 mg of solid samples of Form CSI were stored under different conditions of 25° C./60% RH, 40° C./75% RH, and 60° C./75% RH. Crystalline form and chemical impurity were checked by XRPD and HPLC, respectively. The results are shown in Table 8 and FIG. 5.

TABLE 8

| Condition | Time | Solid Form | Purity (%) |
|---|---|---|---|
| Initial | — | Form CSI | 99.71 |
| 25° C./60% RH (sealed) | 6 Months | Form CSI | 99.65 |
| 25° C./60% RH (open) | 6 Months | Form CSI | 99.66 |
| 40° C./75% RH (sealed) | 6 Months | Form CSI | 99.64 |
| 40° C./75% RH (open) | 6 Months | Form CSI | 99.65 |
| 60° C./75% RH (sealed) | 1 Month | Form CSI | 99.70 |

The results show that Form CSI is stable for at least six months at 25° C./60% RH and 40° C./75% RH. Form CSI has good stability under both long-term and accelerated conditions. Form CSI is stable for at least one month at 60° C./75% RH. Form CSI has good stability under more stress condition.

### Example 5: Humidity Stability of Form CSI

DVS was applied to test the stability of Form CSI under different humidity with about 10 mg of samples. The weight change at each relative humidity were recorded in a humidity range of 0-95% RH. The results are shown in Table 9.

TABLE 9

| Form | Weight loss |
|---|---|
| prior art Form A | 2.7% (30%-5% RH) |
| Form CSI | 0.21% (30-0% RH) |

The weight loss of prior art Form A under 30%-5% RH is 2.7% and Form A converted to dehydrated state Form C under 5% RH. The weight loss of Form CSI of the present disclosure under 30%-0% RH is only 0.21%, which is much lower than that of prior art Form A, indicating that Form CSI of the present disclosure has less weight change in lower humidity, and has better stability under low humidity.

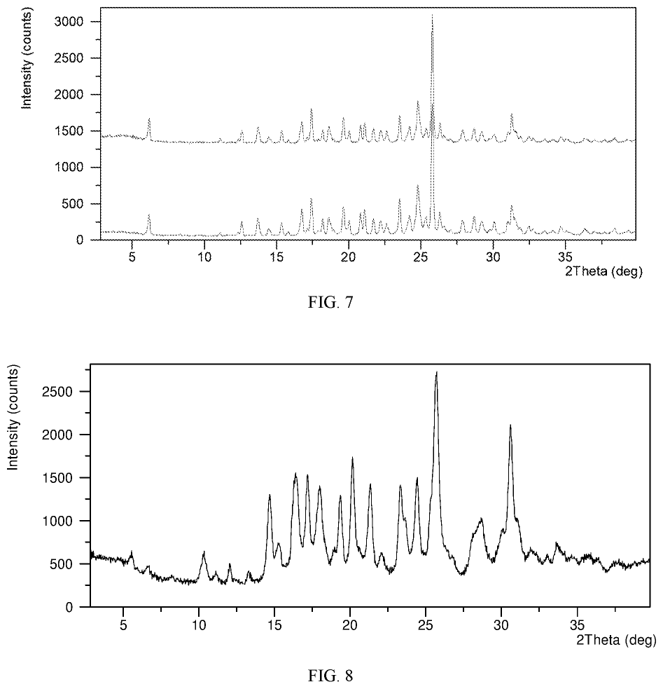

The DVS plot of Form CSI is shown in FIG. 6 and the XRPD pattern overlay of Form CSI before and after DVS test is shown in FIG. 7. The results show that the crystalline state of Form CSI remains unchanged after DVS, which indicates that Form CSI has good humidity stability.

### Example 6: Preparation of Form CSIII

98.5 mg of Compound I dihydrochloride, 63.8 mg of L-tartaric acid, and 5.0 mL of ethyl acetate were mixed and the obtained suspension was stirred at room temperature for 18 days. Then 5.0 mL of ethyl acetate was added into the system and the system was stirred at room temperature for 14 days. The solid was separated and dried at 50° C. for 2.5 hours. The obtained solid was confirmed to be Form CSIII, and the XRPD pattern of Form CSIII is substantially as depicted in FIG. 8, and the XRPD data are listed in Table 10.

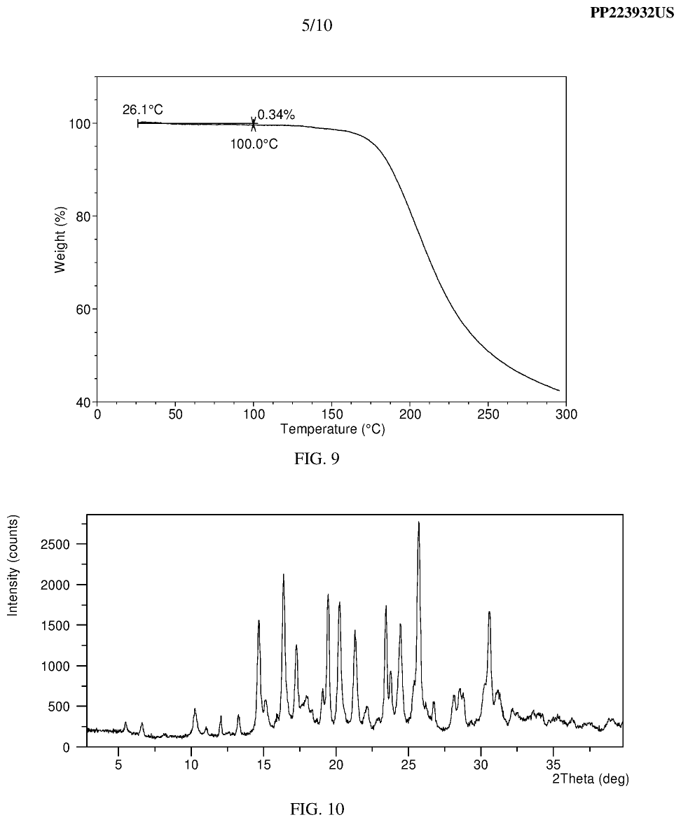

The TGA curve of Form CSIII shows about 0.3% weight loss when heated to 100° C., which is substantially as depicted in FIG. 9.

The assay of Compound I, chloride ion and tartaric acid in Form CSIII was determined by HPLC and IC. The test results show that the molar ratio of chloride ion and Compound I in Form CSIII is 2:1, and the molar ratio of Compound I and tartaric acid is 1:1. The results are shown in Table 11.

TABLE 10

| 2θ (°) | d spacing (Å) | Relative Intensity % |
|---|---|---|
| 5.56 | 15.91 | 6.57 |
| 6.65 | 13.29 | 3.48 |
| 8.17 | 10.82 | 1.33 |
| 10.33 | 8.57 | 13.55 |
| 11.12 | 7.96 | 4.28 |
| 12.06 | 7.34 | 9.31 |
| 13.29 | 6.66 | 5.60 |
| 14.69 | 6.03 | 43.08 |
| 15.28 | 5.80 | 18.86 |
| 16.42 | 5.40 | 52.75 |
| 17.19 | 5.16 | 52.44 |
| 17.99 | 4.93 | 46.16 |
| 19.37 | 4.58 | 41.35 |
| 20.16 | 4.40 | 60.65 |
| 21.33 | 4.16 | 47.06 |
| 22.07 | 4.03 | 12.41 |
| 23.33 | 3.81 | 45.52 |
| 23.68 | 3.76 | 27.60 |
| 24.42 | 3.65 | 48.43 |
| 25.70 | 3.47 | 100.00 |
| 28.07 | 3.18 | 18.38 |
| 28.69 | 3.11 | 26.69 |
| 30.59 | 2.92 | 74.23 |
| 32.05 | 2.79 | 10.19 |
| 33.03 | 2.71 | 7.52 |
| 33.71 | 2.66 | 12.68 |
| 35.66 | 2.52 | 6.68 |
| 36.34 | 2.47 | 5.32 |
| 37.62 | 2.39 | 2.48 |

TABLE 11

| Chloride ion:Compound I | Compound I:Tartaric acid |
|---|---|
| 2.006:1 | 1.001:1 |

### Example 7: Preparation of Form CSIII

587.4 mg of Compound I dihydrochloride solid, 384.6 mg of L-tartaric acid and 20 mL of ethyl acetate were mixed and stirred at room temperature for 11 days. The solid was separated and dried under vacuum at 50° C. for 2.5 hours. The obtained dried solid was further mixed with 13 mL of ethyl acetate and stirred at room temperature for 1 day. The solid was separated and dried under vacuum at 40° C. for about 2 hours. The obtained solid was confirmed to be Form CSIII, and the XRPD pattern of Form CSIII is substantially as depicted in FIG. 10, and the XRPD data are listed in Table 12.

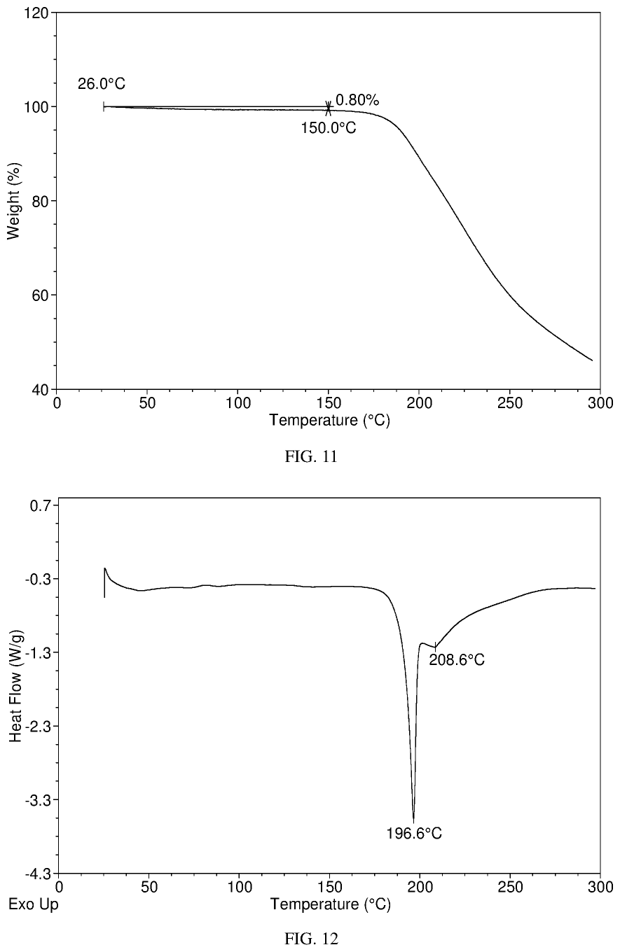

The TGA curve of Form CSIII shows about 0.8% weight loss when heated to 150° C., which is substantially as depicted in FIG. 11.

The DSC curve of Form CSIII is substantially as depicted in FIG. 12, which shows two endothermic peaks at around 197° C. and 209° C.

TABLE 12

| 2θ (°) | d spacing (Å) | Relative Intensity % |
|---|---|---|
| 5.50 | 16.07 | 5.25 |
| 6.61 | 13.38 | 5.59 |
| 10.27 | 8.61 | 12.07 |
| 11.05 | 8.01 | 4.05 |
| 12.07 | 7.33 | 9.73 |
| 13.28 | 6.67 | 9.77 |
| 14.68 | 6.04 | 54.19 |
| 15.14 | 5.85 | 17.02 |
| 16.38 | 5.41 | 75.01 |
| 17.27 | 5.13 | 42.34 |
| 17.99 | 4.93 | 18.38 |
| 18.32 | 4.84 | 12.12 |
| 19.08 | 4.65 | 22.23 |
| 19.46 | 4.56 | 65.74 |
| 20.24 | 4.39 | 62.10 |
| 21.32 | 4.17 | 48.88 |
| 22.16 | 4.01 | 13.95 |
| 23.44 | 3.80 | 60.58 |
| 23.77 | 3.74 | 30.59 |
| 24.45 | 3.64 | 52.36 |
| 25.31 | 3.52 | 21.59 |
| 25.71 | 3.46 | 100.00 |
| 26.75 | 3.33 | 16.35 |
| 28.14 | 3.17 | 18.98 |
| 28.53 | 3.13 | 22.25 |
| 28.76 | 3.10 | 19.96 |
| 30.18 | 2.96 | 22.10 |
| 30.58 | 2.92 | 58.06 |
| 31.13 | 2.87 | 21.02 |
| 32.16 | 2.78 | 12.89 |
| 34.07 | 2.63 | 9.96 |
| 35.21 | 2.55 | 8.64 |
| 36.29 | 2.48 | 8.10 |
| 37.39 | 2.41 | 6.09 |
| 38.96 | 2.31 | 7.49 |

As disclosed in WO2014152270A1, the prior art Form A began losing weight at room temperature in the heating process, and there is a weight loss of about 2 to 5% in the range of about 100° C. to about 150° C., and when heated to 75-100° C., Form A converts to Form B. While Form CSIII of the present disclosure only has a mass change of 0.80% when heated to 150° C., and there is no thermal signal in DSC process before 150° C., indicating that Form CSIII has no crystal transformation before 150° C. and has better stability at higher temperature (below 150° C.), which is more beneficial for the stability of formulation processing and industrial production.

### Example 8: Stability of Form CSIII

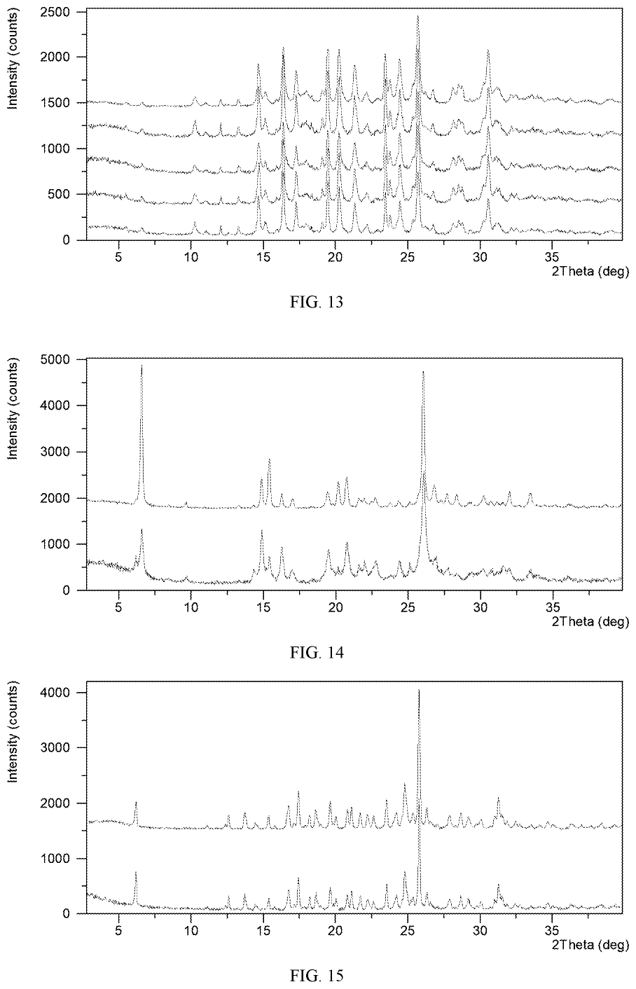

Approximately 5 mg of solid samples of Form CSIII were stored under different conditions of 25° C./60% RH, 40° C./75% RH, and 60° C./75% RH. Crystalline form and chemical impurity were checked by XRPD and HPLC, respectively. The results are shown in Table 13 and FIG. 13.

TABLE 13

| Condition | Time | Solid Form | Purity (%) |
|---|---|---|---|
| Initial | — | Form CSIII | 99.32 |
| 25° C./60% RH (sealed with | 3 Months | Form CSIII | 99.31 |
| desiccant) | | | |
| 25° C./60% RH (open) | 3 Months | Form CSIII | 99.35 |
| 40° C./75% RH (sealed with | 3 Months | Form CSIII | 99.32 |
| desiccant) | | | |
| 60° C./75% RH (sealed with | 3 Months | Form CSIII | 99.36 |
| desiccant) | | | |

The results show that Form CSIII is stable for at least 3 months at 25° C./60% RH and 40° C./75% RH. Form CSIII has good stability under both long-term and accelerated conditions. Form CSIII is stable for at least 3 months at 60° C./75% RH. Form CSIII has good stability under more stress condition.

### Example 9: Humidity Stability of Form CSIII

DVS was applied to test the stability of Form CSIII under different humidity with about 10 mg of samples. The weight change at each relative humidity were recorded in a humidity range of 0-95% RH.

The weight loss of prior art Form A under 30%-5% RH is 2.7% and it converted to Form C under 5% RH. The weight loss of Form CSIII of the present disclosure under 30%-0% RH is only 1.81%. The results indicate that Form CSIII of the present disclosure has less weight change in a lower humidity range and has better stability under low humidity.

### Example 10: Compressibility of CSIII

A manual tablet press was used for compression. 60 mg of Form CSIII and prior art Form A were weighed and added into the dies of a φ6 mm round tooling, compressed at 10 KN manually, then stored at room temperature for 24 h until complete elastic recovery, diameter (D) and thickness (L) were tested with a caliper. Hardness (H) was tested with an intelligent tablet hardness tester. Tensile strength of the powder was calculated with the following formula: T=2H/πDL. Under a certain force, the greater the tensile strength, the better the compressibility. The results are presented in Table 14.

TABLE 14

| | Thickness | Diameter | Hardness | Tensile strength |
|---|---|---|---|---|
| Form | (mm) | (mm) | (kgf) | (MPa) |
| Form A | 1.67 | 6.00 | 3.38 | 2.11 |
| Form CSIII | 1.62 | 6.00 | 3.51 | 2.26 |

The results indicate that Form CSIII has better compressibility compared with prior art Form A.

### Example 11: Mechanical Stability of Form CSI and Form CSIII

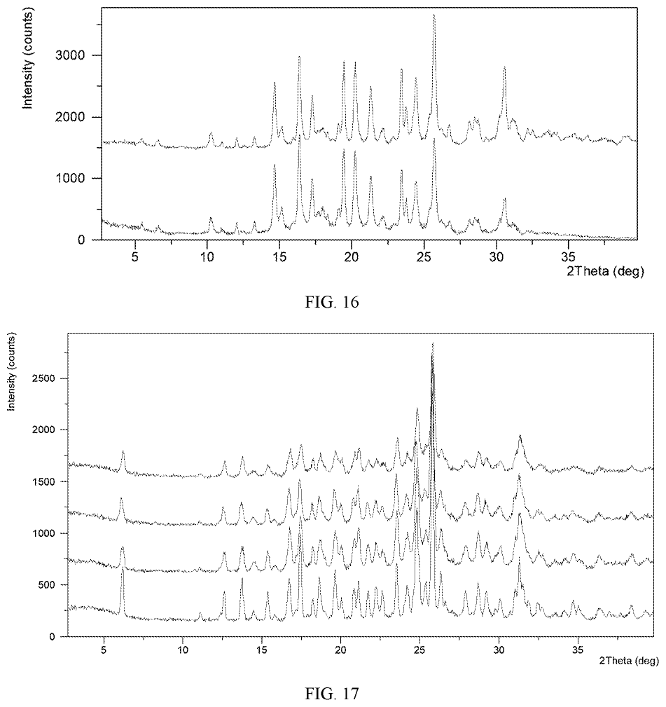

Solid sample of prior art Form A, Form CSI and Form CSIII of the present disclosure were separately grounded manually for 5 minutes in mortars. The XRPD patterns overlay before and after grinding is shown in FIG. 14, FIG. 15 and FIG. 16.

The results show that the crystallinity of prior art Form A decreases after grinding, while Form CSI and Form CSIII of the present disclosure have no crystal transformation and the crystallinity almost has no change after grinding, which indicates that Form CSI and Form III have better grinding stability when compared with Form A in the prior art.

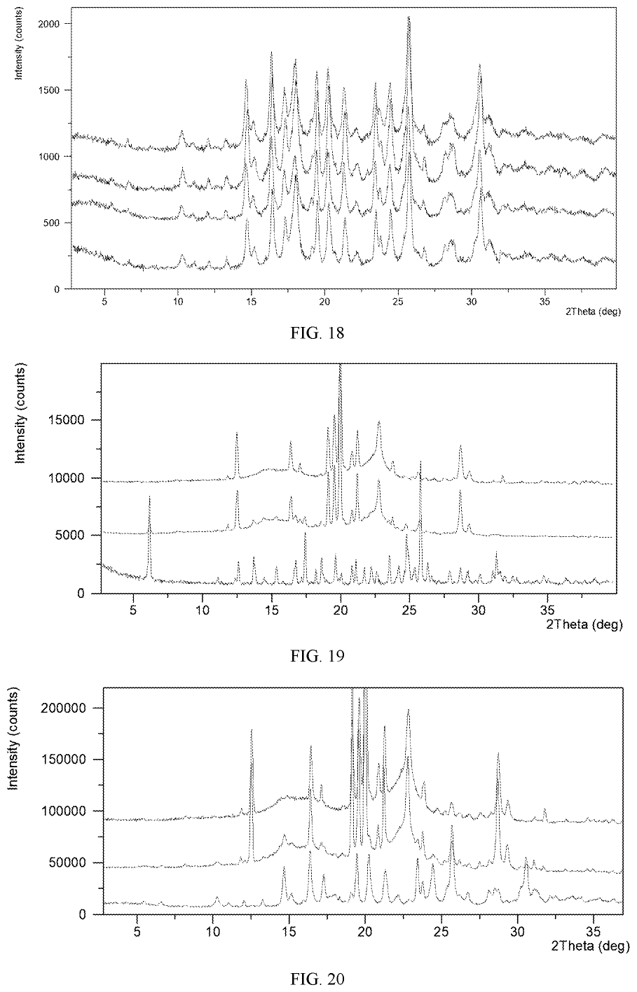

A certain amount of Form CSI and Form CSIII were compressed into tablets under 5 kN, 10 kN, 20 kN pressure with suitable tableting die. Crystalline form before and after tableting were checked by XRPD. The results show that Form CSI and Form CSIII have no crystal transformation after tableting under different pressure. The XRPD pattern overlays are shown in FIG. 17 and FIG. 18.

### Example 12: Adhesiveness of Form CSI

30 mg of Form CSI, Form CSIII and prior art Form A were weighed and then added into the dies of φ8 mm round tooling, compressed at 10 KN and held for 30 s. The punch was weighed and the amount of material sticking to the punch was calculated. The compression was repeated twice and the maximum amount of material sticking to the punch during the compression were recorded. Detailed experimental results are shown in Table 15. Test results indicate that the adhesiveness of Form CSI and Form CSIII of the present disclosure is superior to the prior art Form A and the maximum amount is less than ⅕ of that of the prior art.

TABLE 15

| Form | Maximum amount (mg) |
|---|---|
| Form A | 2.83 |
| Form CSI | 0.48 |
| Form CSIII | 0.40 |

### Example 13: Preparation of Form CSI and Form CSIII Drug Product

The formulation and preparation process of Form CSI and Form CSIII are shown in Table 16 and Table 17, respectively. The XRPD overlay of Form CSI and Form CSIII before and after formulation process are shown in FIG. 19 and FIG. 20. The results show that Form CSI and Form CSIII remain stable before and after the formulation process.

TABLE 16

| Form | | Form CSI | | Form CSIII | | |
|---|---|---|---|---|---|---|
| No. | Component | mg/unit | % (w/w) | mg/unit | % (w/w) | Function |
| 1 | Drug substance* | 34.01 | 13.60 | 38.98 | 15.59 | API |
| 2 | Fumaric acid | 34.01 | 13.60 | 38.98 | 15.59 | pH regulator |
| 3 | Microcrystalline | 88.23 | 35.29 | 78.29 | 31.32 | Filler |
| | Cellulose (PH102) | | | | | |
| 4 | Lactose monohydrate | 75.00 | 30.00 | 75.00 | 30.00 | Filler |
| | (Armor Pharma 150 | | | | | |
| | mesh) | | | | | |
| 5 | Hydroxypropyl | 5.00 | 2.00 | 5.00 | 2.00 | Binder |
| | methylcellulose | | | | | |
| | (EXF) | | | | | |
| 6 | Croscarmellose | 6.25 | 2.50 | 6.25 | 2.50 | Disintegrant |
| | sodium | | | | | |
| Subtotal | | 242.50 | 97.00 | 242.50 | 97.00 | N/A |
| 7 | Croscarmellose | 6.25 | 2.50 | 6.25 | 2.50 | Disintegrant |
| | sodium | | | | | |
| 8 | Magnesium stearate | 1.25 | 0.50 | 1.25 | 0.50 | Lubricant |
| | (5712) | | | | | |

| | | | | | |
|---|---|---|---|---|---|
| Total | 250 | 100 | 250 | 100 | N/A |

*The sample weight is calculated with the consideration of the molecular weight and TGA weight loss of different APIs and each tablet corresponds to 25 mg compound I.

TABLE 17

| Stage | Procedure |
|---|---|
| Pre-blending | According to the formulation, materials No. 1-6 were |
| | weighed into an LDPE bag and blended for 2 mins. |
| Simulation | The mixture was pressed by a single punch manual |
| of dry | tablet press (type: ENERPAC; die: φ 20 mm round; |
| granulation | flake weight: 500 mg ± 100 mg; pressure: 5 ± 1 KN) |
| | and flakes were obtained. The flakes were pulverized |
| | and sieved through a 20-mesh sieve. |
| Final blending | Materials No. 7-8 were weighed and added into an |
| | LDPE bag together with the flakes after dry |
| | granulation and the mixture was blended for 2 mins. |
| Tableting | The mixture was tableted by a single punch manual |
| | tablet press (type: ENERPAC; die: φ9 mm round; |
| | tablet weight: 250 mg ± 10 mg; pressure: 7 ± |
| | 1 KN) |

### Example 14 Stability of Form CSI and Form CSIII in Drug Product

The drug products of Form CSI and Form CSIII prepared according to Example 13 were stored under 40° C./75% RH condition. The chemical impurity and crystalline form of the sample were tested by HPLC and XRPD, respectively. The stability results of the Form CSI and Form CSIII drug products are shown in Table 18.

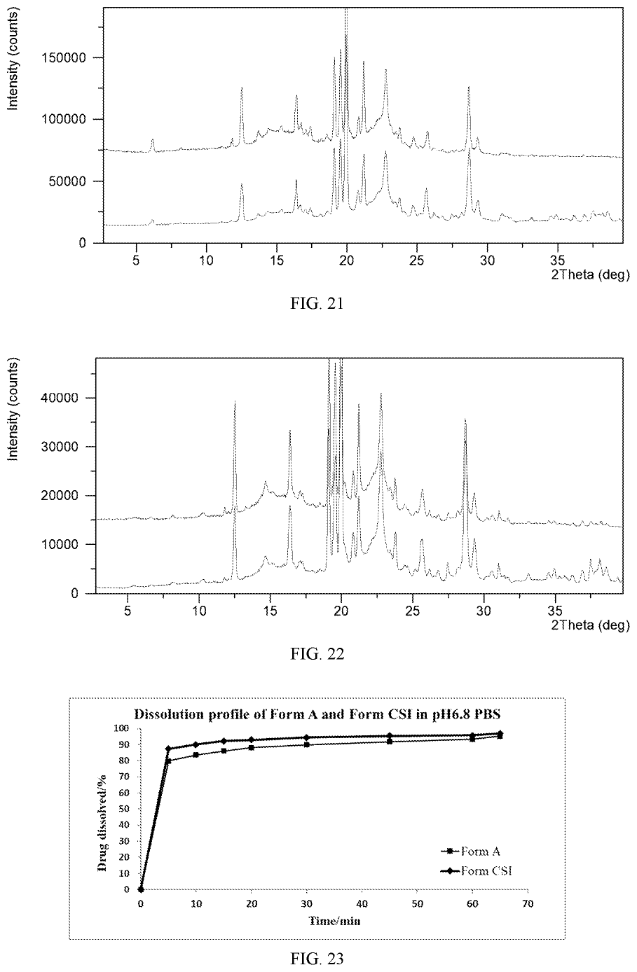

TABLE 18

| Sample | Time | Form | Purity (% ) | FIG. |
|---|---|---|---|---|
| Form CSI tablet | Initial | Form CSI | 99.69 | FIG. 21 |
| | 3 Months | Form CSI | 99.71 | |
| Form CSI tablet | Initial | Form CSIII | 99.31 | FIG. 22 |
| | 3 Months | Form CSIII | 99.35 | |

| | |
|---|---|
| Packing Condition | 35 cc HDPE bottle + 1 g desiccant |

The results indicate that Form CSI and Form CSIII drug products can keep physically and chemically stable under 40° C.±2° C./75%±5% RH for at least 3 months and the chemical purity remains substantially unchanged.

### Example 15: Dissolution of Form CSI Drug Product

Dissolution test was performed on Form CSI and prior art Form A drug product obtained from example 13. Dissolution method according to Chinese Pharmacopoeia 2020<0931>was used. The conditions are shown in Table 19.

TABLE 19

| | |
|---|---|
| Dissolution tester | Agilent 708DS |
| Method | Paddle |
| Strength | 25 mg |
| Volume | 900 mL |
| Speed | 50 rpm |
| Temperature | 37° C. |
| Time | 5, 10, 15, 20, 30, 45, 60 min |
| Supplementary medium | No |

Dissolution results of Form CSI and prior art Form A drug products are presented in Table 20, the dissolution curves are shown in FIG. 23, which indicate that Form CSI drug product possesses better dissolution.

TABLE 20

Medium
pH 6.8 PBS

| Time (min) | Form A | Form CSI |
|---|---|---|
| 0 | 0.0 | 0.0 |
| 5 | 79.9 | 87.4 |
| 10 | 83.5 | 90.0 |
| 15 | 86.1 | 92.2 |
| 20 | 88.1 | 92.9 |
| 30 | 89.9 | 94.5 |
| 45 | 91.8 | 95.4 |
| 60 | 93.4 | 95.9 |

The examples described above are only for illustrating the technical concepts and features of the present disclosure, and intended to make those skilled in the art being able to understand the present disclosure and thereby implement it, and should not be concluded to limit the protective scope of this disclosure. Any equivalent variations or modifications according to the spirit of the present disclosure should be covered by the protective scope of the present disclosure.

What is claimed is:

1. A co-crystal of Compound I dihydrochloride with fumaric acid

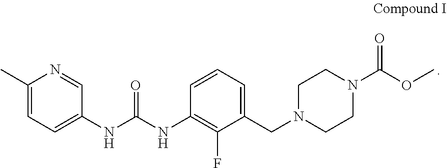

2. The co-crystal of Compound I dihydrochloride with fumaric acid according to claim 1, wherein the molar ratio of Compound I dihydrochloride and fumaric acid is 2:1.

3. The co-crystal of Compound I dihydrochloride with fumaric acid according to claim 1, wherein the X-ray powder diffraction pattern comprises characteristic peaks at 2theta values of 6.2°±0.2°, 17.4°±0.2° and 25.8°±0.2° using CuKα radiation.

4. The co-crystal of Compound I dihydrochloride with fumaric acid according to claim 3, wherein the X-ray powder diffraction pattern comprises one or two or three characteristic peaks at 2theta values of 12.6°±0.2°, 19.6°±0.2° and 23.5°±0.2° using CuKα radiation.

5. The co-crystal of Compound I dihydrochloride with fumaric acid according to claim 3, wherein the X-ray powder diffraction pattern comprises one or two or three characteristic peaks at 2theta values of 15.4°±0.2°, 21.1°±0.2° and 26.3°±0.2° using CuKα radiation.

6. A process for preparing the co-crystal of Compound I dihydrochloride with fumaric acid according to claim 1, wherein the process comprises: adding Compound I dihydrochloride solid and fumaric acid solid into a solvent mixture of a nitrile and water, stirring to obtain the co-crystal of Compound I dihydrochloride with fumaric acid.

7. The process according to claim 6, wherein the molar ratio of said Compound I dihydrochloride solid and fumaric acid solid is 1:3-2:1, said nitrile is acetonitrile, and volume ratio of acetonitrile and water in said solvent mixture is 9:1.

8. A co-crystal of Compound I dihydrochloride with tartaric acid

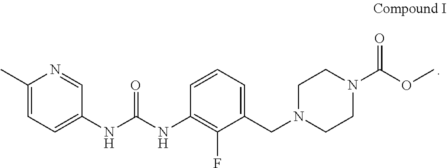

9. The co-crystal of Compound I dihydrochloride with tartaric acid according to claim 8, wherein the molar ratio of Compound I dihydrochloride and tartaric acid is 1:1.

10. The co-crystal form of Compound I dihydrochloride with tartaric acid according to claim 8, wherein the X-ray powder diffraction pattern comprises characteristic peaks at 2theta values of 17.2°±0.2°, 20.2°±0.2° and 25.7°±0.2° using CuKα radiation.

11. The co-crystal of Compound I dihydrochloride with tartaric acid according to claim 10, wherein the X-ray powder diffraction pattern comprises one or two or three characteristic peaks at 2theta values of 19.4°±0.2°, 24.4°±0.2° and 30.6°±0.2° using CuKα radiation.

12. The co-crystal of Compound I dihydrochloride with tartaric acid according to claim 10, wherein the X-ray powder diffraction pattern comprises one or two or three characteristic peaks at 2theta values of 18.0°±0.2°, 14.7°±0.2° and 21.3°±0.2° using CuKα radiation.

13. A process for preparing the co-crystal of Compound I dihydrochloride with tartaric acid according to claim 8, wherein the process comprises: adding Compound I dihydrochloride solid and tartaric acid solid in an ester, slurring and separating to obtain the co-crystal of Compound I dihydrochloride with tartaric acid.

14. The process according to claim 13, wherein the molar ratio of said Compound I dihydrochloride solid and tartaric acid solid is 1:3-1:1, and said ester is ethyl acetate.

15. A pharmaceutical composition, wherein said pharmaceutical composition comprises a therapeutically effective amount of the co-crystal of Compound I dihydrochloride with fumaric acid according to claim 1 and pharmaceutically acceptable excipients.

16. A pharmaceutical composition, wherein said pharmaceutical composition comprises a therapeutically effective amount of the co-crystal of Compound I dihydrochloride with tartaric acid according to claim 8 and pharmaceutically acceptable excipients.

17. A method of agonizing cardiac muscle myosin, comprising administering to a subject in need thereof a therapeutically effective amount of the co-crystal of Compound I dihydrochloride with fumaric acid according to claim 1.

18. A method of agonizing cardiac muscle myosin, comprising administering to a subject in need thereof a therapeutically effective amount of the co-crystal of Compound I dihydrochloride with tartaric acid according to claim 8.

19. A method for treating heart failure, comprising administering to a subject in need thereof a therapeutically effective amount of the co-crystal of Compound I dihydrochloride with fumaric acid according to claim 1.

20. A method for treating heart failure, comprising administering to a subject in need thereof a therapeutically effective amount of the co-crystal of Compound I dihydrochloride with tartaric acid according to claim 8.
